(19) United States
(12) Patent Application Publication
(10) Pub. No.: US 2010/0036423 A1
(43) Pub. Date: Feb. 11, 2010

(54) Dynamic rod

(76) Inventors: Stanley Kyle Hayes, Mission Viejo, CA (US); Joey Camia Reglos, Lake Forest, CA (US); Moti Altarac, Irvine, CA (US); Daniel H. Kim, Houston, TX (US)

Correspondence Address: RIMAS LUKAS;VERTIFLEX, INC., 1351 CALLE AVANZADO, SAN CLEMENTE, CA, 92673, US

(21) Appl. No.: 12/540,865

(22) Filed: Aug. 13, 2009

Related U.S. Application Data
(63) Continuation-in-part of application No. 12/154,540, filed on May 23, 2008.
Application No. 12/154,540 is a continuation-in-part of application No. 12/233,212, filed on Sep. 18, 2008.
Application No. 12/233,212 is a continuation-in-part of application No. 12/366,089, filed on Feb. 5, 2009.
Application No. 12/366,089 is a continuation-in-part of application No. 11/427,738, filed on Jun. 29, 2006.
Application No. 11/427,738 is a continuation-in-part of application No. 11/436,407, filed on May 17, 2006.
Application No. 11/436,407 is a continuation-in-part of application No. 11/033,452, filed on Jan. 10, 2005.
Application No. 11/033,452 is a continuation-in-part of application No. 11/006,495, filed on Dec. 6, 2004.
Application No. 11/006,495 is a continuation-in-part of application No. 10/970,366, filed on Oct. 20, 2004.
(60) Provisional application No. 61/188,976, filed on Aug. 14, 2008.
(60) Provisional application No. 60/931,811, filed on May 25, 2007.
(60) Provisional application No. 60/994,899, filed on Sep. 21, 2007.
(60) Provisional application No. 61/063,878, filed on Feb. 6, 2008.

(51) Int. Cl. A61B 17/70

(52) U.S. Cl. 606/260

(57) ABSTRACT

A dynamic rod implantable into a patient and connectable between two vertebral anchors in adjacent vertebral bodies is provided. The dynamic rod fixes the adjacent vertebral bodies together in a dynamic fashion providing immediate postoperative stability and support of the spine. The dynamic rod comprises a first rod portion dynamically connected to a second rod portion at a retainer that has separate chambers for receiving rod portions. One rod portion is configured for longitudinal movement and the other rod portion is configured for polyaxial angulation relative to the retainer. The dynamic rod is configured such that the retainer is located proximate to one of the facet joints when implanted into a patient. The dynamic rod permits relative movement of the first and second rod portions allowing the rod to carry some of the natural flexion, extension and rotation moments of the spine.

### CROSS-REFERENCE TO RELATED APPLICATIONS

This application claims priority to and the benefit of U.S. Provisional Patent Application Ser. No. 61/188,976 entitled “Dynamic rod” filed on Aug. 14, 2008 which is incorporated herein by reference in its entirety. This application also claims priority to and is a continuation-in-part of co-pending U.S. patent application Ser. No. 12/154,540 entitled “Dynamic rod” filed on May 23, 2008 which claims the benefit of U.S. Provisional Patent Application Ser. No. 60/931,811 filed on May 25, 2007. This application is also a continuation-in-part of co-pending U.S. patent application Ser. No. 12/233,212 entitled “Dynamic rod” filed on Sep. 18, 2008 which claims the benefit of U.S. Provisional Patent Application Ser. No. 60/994,899 entitled “Dynamic rod” filed on Sep. 21, 2007. This application is also a continuation-in-part of co-pending U.S. patent application Ser. No. 12/366,089 entitled “Dynamic rod” filed on Feb. 5, 2009 which claims the benefit of U.S. Provisional Patent Application Ser. No. 61/063,878 entitled “Dynamic rod” filed on Feb. 6, 2008. This application is also a continuation-in-part of co-pending U.S. patent application Ser. No. 11/427,738 entitled “Systems and methods for stabilization of the bone structures” filed on Jun. 29, 2006 which is a continuation-in-part of U.S. patent application Ser. No. 11/436,407 entitled “Systems and methods for stabilization of the bone structures” filed on May 17, 2006 which is a continuation-in-part of U.S. patent application Ser. No. 11/033,452 entitled “Systems and methods for stabilization of the bone structures” filed on Jan. 10, 2005 which is a continuation-in-part of U.S. patent application Ser. No. 11/006,495 entitled “Systems and methods for stabilization of the bone structures” filed on Dec. 6, 2004 which is a continuation-in-part of U.S. patent application Ser. No. 10/970,366 entitled “Systems and methods for stabilization of the bone structures” filed on Oct. 20, 2004. All of the above-referenced applications are each incorporated herein by reference in their entirety.

### FIELD

The present invention generally relates to devices, systems, and methods for the fixation of the spine. In particular, the present invention relates to a system applied posteriorly to the spine that provides dynamic support to spinal vertebrae and controls load transfers to avoid deterioration of the vertebral disc or bone of adjacent spinal vertebrae.

### BACKGROUND

Damage to the spine as a result of advancing age, disease, and injury, has been treated in many instances by fixation or stabilization of vertebrae. Conventional methods of spinal fixation utilize a rigid spinal fixation device to support an injured spinal vertebra relative to an adjacent vertebra and prevent movement of the injured vertebra relative to an adjacent vertebra. These conventional spinal fixation devices include anchor members for fixing to a series of vertebrae of the spine and at least one rigid link element designed to interconnect the anchor members. Typically, the anchor member is a screw and the rigid link element is a rod. The screw is configured to be inserted into the pedicle of a vertebra to a predetermined depth and angle. One end of the rigid link element is connected to an anchor inserted in the pedicle of the upper vertebra and the other end of the rod is connected to an anchor inserted in the pedicle of an adjacent lower vertebra. The rod ends are connected to the anchors via coupling constructs such that the adjacent vertebrae are supported and held apart in a relatively fixed position by the rods. Typically, two rods and two pairs of anchors are installed each in the manner described above such that two rods are employed to fix two adjacent vertebrae, with one rod positioned on each side of adjacent vertebrae. Once the system has been assembled and fixed to a series of two or more vertebrae, it constitutes a rigid device preventing the vertebrae from moving relative to one another. This rigidity enables the devices to support all or part of the stresses instead of the stresses being born by the series of damaged vertebra.

While these conventional procedures and devices have been proven capable of providing reliable fixation and stabilization of the spine, the resulting constructs typically provide a very high degree of rigidity to the operative levels of the spine resulting in decreased mobility of the vertebral segment. Unfortunately, this high degree of rigidity imparted to the spine by such devices can sometimes be excessive. Because the patient's fixed vertebrae are not allowed to move, the vertebrae located adjacent to, above or below, the series that has undergone such fixation tend to move more in order to compensate for the decreased mobility. As a result, a concentration of additional mechanical stresses is placed on these adjacent vertebral levels and a sharp discontinuity in the distribution of stresses along the spine can then arise between, for example, the last vertebra of the series and the first free vertebra. This increase in stress can accelerate degeneration of the vertebrae at these adjacent levels.

Sometimes, fixation accompanies a fusion procedure in which bone growth is encouraged to bridge the intervertebral body disc space to thereby fuse adjacent vertebrae together. Fusion involves removal of a damaged intervertebral disc and introduction of an interbody spacer along with bone graft material into the intervertebral disc space. In cases where fixation accompanies fusion, excessively rigid spinal fixation is not helpful to the promotion of the fusion process due to load shielding away from the fixed series. Without the stresses and strains, bone does not have loads to adapt to and as bone loads decrease, the bone becomes weaker. Thus, fixation devices that permit load sharing and assist the bone fusion process are desired in cases where fusion accompanies fixation.

Various improvements to fixation devices such as a link element having a dynamic central portion have been devised. These types of dynamic rods support part of the stresses and help relieve the vertebrae that are overtaxed by fixation. Some dynamic rods are designed to permit axial load transmission substantially along the vertical axis of the spine to prevent load shielding and promote the fusion process. Dynamic rods may also permit a bending moment to be partially transferred by the rod to the fixed series that would otherwise be born by vertebrae adjacent to the fixed series. Compression or extension springs can be coiled around the rod for the purpose of providing de-rotation forces as well as relative translational sliding movement along the vertical axis of the spine. Overall, the dynamic rod in the fixation system plays an important role in recreating the biomechanical organization of the functional unit made up of two fixed vertebrae together with the intervertebral disc.

In conclusion, conventional spinal fixation devices have not provided a comprehensive solution to the problems associated with curing spinal diseases in part due to the difficulty of creating a system that mimics a healthy functioning spinal unit. Hence, there is a need for an improved dynamic spinal fixation device that provides a desired level of flexibility to the fixed series of the spinal column, while also providing long-term durability and consistent stabilization of the spinal column.

### SUMMARY

According to one aspect of the invention, a dynamic rod is provided. The dynamic rod includes a first rod portion dynamically connected to a second rod portion dynamically connected to the first rod portion at a retainer. The retainer includes a first chamber configured to retain an end of the first rod portion and a second chamber configured to retain an end of the second rod portion and the first and second chambers are separated by a wall formed in the retainer.

According to another aspect of the invention, a dynamic rod is provided. The dynamic rod includes a first rod portion dynamically connected to a second rod portion at a retainer. The retainer is configured to retain an end of the first rod portion and an end of the second rod portion. The dynamic rod further includes a pin passed through at least one slot formed in the retainer and connected to the first rod portion such that longitudinal movement of the first rod portion relative to the retainer is limited to within the at least one slot.

According to another aspect of the invention, a dynamic rod is provided. The dynamic rod includes a first rod portion dynamically connected to a second rod portion at a retainer. The retainer is configured to retain an end of the first rod portion and an end of the second rod portion. The first rod portion is configured for longitudinal movement relative to the retainer and the second rod portion is configured to angulate polyaxially relative to the retainer.

According to another aspect of the invention a dynamic rod is provided. The dynamic rod includes a first rod portion dynamically connected to a second rod portion at a retainer. The retainer is configured to retain an end of the first rod portion and an end of the second rod portion. The first rod portion is longer than the second rod portion.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1a illustrates a perspective view of a dynamic rod according to the present invention.

FIG. 1b illustrates a side view of a dynamic rod of FIG. 1a according to the present invention.

FIG. 1c illustrates a cross-sectional view of the dynamic rod of FIG. 1a taken along line A-A of FIG. 1b according to the present invention.

FIG. 2a illustrates a perspective view of a first rod portion of a dynamic rod according to the present invention.

FIG. 2b illustrates a side view of the first rod portion of FIG. 2a of a dynamic rod according to the present invention.

FIG. 2c illustrates a cross-sectional view of the first rod portion taken along line A-A of FIG. 2b of a dynamic rod according to the present invention.

FIG. 2d illustrates a side view of a pin of a first rod portion of a dynamic rod according to the present invention.

FIG. 3a illustrates a perspective view of a second rod portion of the dynamic rod according to the present invention.

FIG. 3b illustrates a side view of the second rod portion of a dynamic rod according to the present invention.

FIG. 4a illustrates a perspective view of a bias element of a dynamic rod according to the present invention.

FIG. 4b illustrates a side view of a bias element of the dynamic rod according to the present invention.

FIG. 4c illustrates a cross-sectional view taken along line A-A of FIG. 4b of a bias element of a dynamic rod according to the present invention.

FIG. 5a illustrates a perspective view of a first piece of a retainer of the dynamic rod of FIG. 1 according to the present invention.

FIG. 5b illustrates a side view of a first piece of a retainer of the dynamic rod according to the present invention.

FIG. 5c illustrates a cross-sectional view taken along line A-A of FIG. 5b of a first piece of a retainer of a dynamic rod according to the present invention.

FIG. 5d illustrates a perspective view of a second piece of a retainer of a dynamic rod according to the present invention.

FIG. 5e illustrates a side view of a second piece of a retainer of a dynamic rod according to the present invention.

FIG. 5f illustrates a cross-sectional view taken along line A-A of FIG. 5e of a second piece of a retainer of a dynamic rod according to the present invention.

FIG. 6a illustrates a side view of a dynamic rod in a contracted state according to the present invention.

FIG. 6b illustrates a side view of a dynamic rod in an extended state according to the present invention.

FIG. 6c illustrates a side view of a dynamic rod with the longitudinal axis depicted in maximum deflected states to illustrate the range of deflection of the dynamic rod according to the present invention.

FIG. 7a illustrates a pair of anchor systems implanted in an upper vertebra and a pair of anchor systems implanted in a lower vertebra of a spine and interconnected by two dynamic rods according to the present invention.

FIG. 7b illustrates a side view of pair of anchor systems implanted in an upper vertebra and a pair of anchor systems implanted in a lower vertebra of a spine and interconnected by two dynamic rods according to the present invention.

FIG. 7c illustrates a posterior view of pair of anchor systems implanted in an upper vertebra and a pair of anchor systems implanted in a lower vertebra of a spine and interconnected by two dynamic rods according to the present invention.

### DETAILED DESCRIPTION

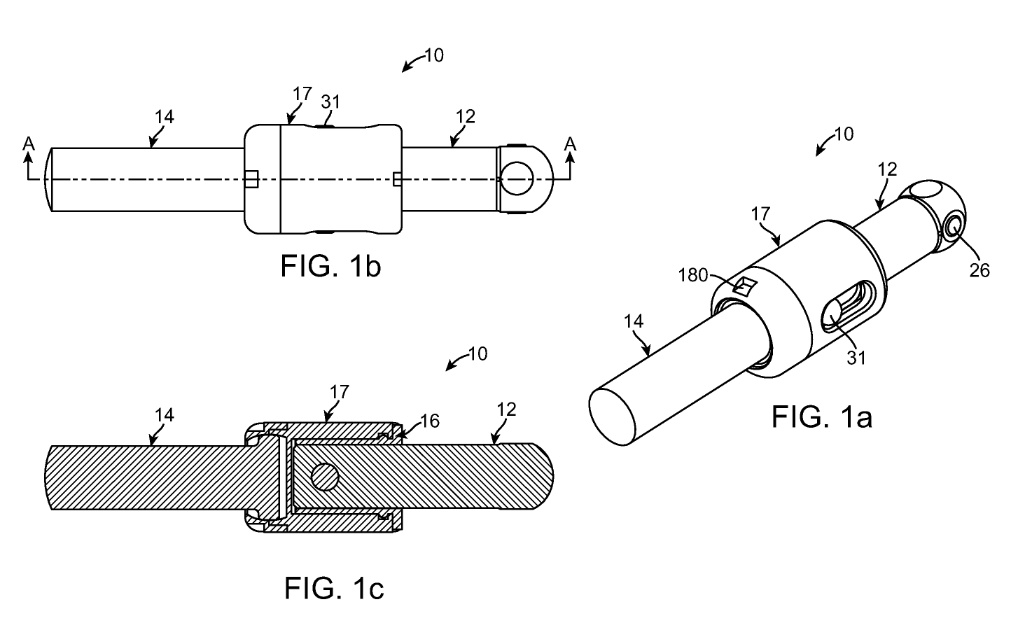

Referring now to FIGS. 1a-1c, there is shown a dynamic rod 10 for use in a spinal fixation system. A spinal fixation system generally includes a first set of two bone anchor systems installed into the pedicles of a superior vertebral segment, a second set of two bone anchor systems installed into the pedicles of an inferior vertebral segment, a first link element connected between one of the pedicle bone anchor systems in the first set and one of the pedicle bone anchor systems in the second set along the same side of the inferior and superior vertebral segments, and a second link element connected between the other of the pedicle bone anchor systems in the first set and the other of the pedicle bone anchor systems in the second set along the same side of the inferior and superior vertebral segments.

A typical anchor system comprises, but is not limited to, a spinal bone screw that is designed to have one end that inserts threadably into a vertebra and a seat polyaxially attached at the opposite end thereof. Typically, the seat is designed to receive the link element in a channel in the seat. The link element is typically a rod or rod-like member. The seat typically has two upstanding arms that are on opposite sides of the channel that receives the rod member. The rod is laid in the open channel, the top of which is then closed with a closure member to both capture the rod in the channel and lock it in the seat to prevent relative movement between the seat and the rod.

With particular reference to FIGS. 1a-1c, a rod 10 according to the present invention comprises a first rod portion 12, a second rod portion 14, a bias element 16, and a retainer 17 or other connecting means for connecting the first and second rod portions 12, 14. The first rod portion 12 is connected to the second rod portion 14 via the retainer 17. The bias element 16 is disposed within the retainer 17 as shown in FIG. 1c.

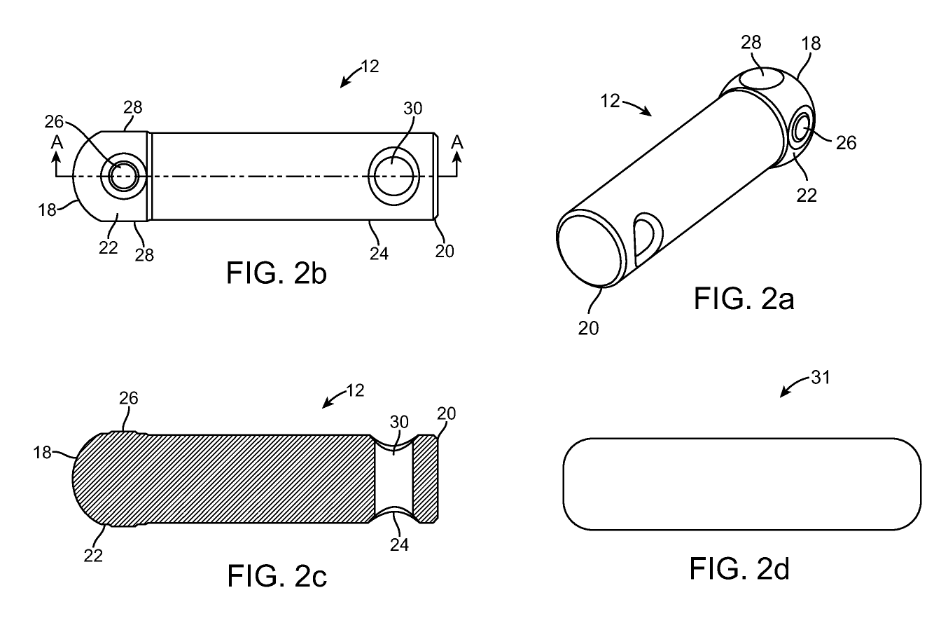

Referring now to FIGS. 2a-2c, the first rod portion 12 includes a first end 18 and a second end 20. The first rod portion 12 is generally cylindrical, elongate and rod-like in shape. An anchor connecting portion 22 is formed at the first end 18 and configured for attachment to an anchor system. The anchor connecting portion 22 shown in FIGS. 2a-2c is partially spherical in shape and includes oppositely disposed outwardly extending pins 26 for engaging slots or apertures formed in the anchor to allow the dynamic rod 10 to snap in and pivot about the pins 26 when connected to the anchor. The anchor connecting portion 22 also includes oppositely disposed flat areas 28. The flat areas 28 are substantially parallel to the longitudinal axis of the pins 26. When the dynamic rod 10 is connected to the anchor and pivoted into a substantially horizontal position, the flat areas 28 face upwardly and downwardly and as a result, provide a lower profile for the rod within the seat of the anchor. Furthermore, the flat areas 28 provide a flat contact surface for a closure member on the upper surface of the rod 10 and a flat contact surface on the bottom surface when seated in the anchor. Although FIGS. 2a-2c shows the rod having an anchor connecting portion 22 configured for a pin-to-slot, snap-fit or compression-fit engagement, the invention is not so limited and any suitable anchor connecting portion configuration is within the scope of the present invention.

Still referencing FIGS. 2a-2c, the first rod portion 12 includes an engaging portion 24 at the second end 20. The engaging portion 24 is configured to engage the retainer 17 of the dynamic rod 10. The engaging portion 24 includes a flat end with beveled edges and a first bore defining a receiving portion 30 for receiving a pin 31. The pin 31 is shown in FIGS. 1a and 2d. The longitudinal axis of the bore is substantially perpendicular to the longitudinal axis of the first rod portion 12. As seen in FIG. 2d, the pin 31 is cylindrical in shape and sized such that when inserted into the first bore 30 the ends of the pin 31 extend beyond the outer surface of the first rod portion 12 to serve as a stop mechanism against the retainer 17 when connected to the retainer 17 so that the first rod portion 12 does not fall out of the retainer 17 as will be discussed further below.

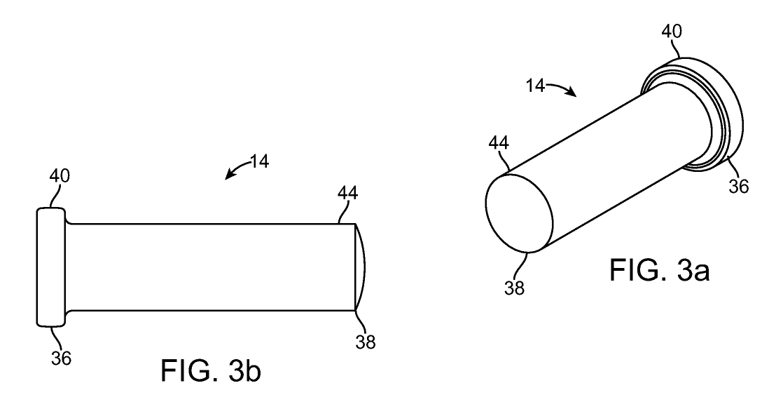

Turning now to FIGS. 3a-3b, there is shown a second rod portion 14. The second rod portion 14 includes a first end 36 and a second end 38. The second rod portion 14 is generally cylindrical, elongate and rod-like in shape and includes an engaging portion 40 at a slightly enlarged and disc-shaped first end 36. The engaging portion 40 is configured to engage with the retainer 17 of the dynamic rod 10. At least a portion of the engaging portion 40 of the second rod portion 14 is configured and sized to fit within the retainer 17 as shown in FIG. 1c. The engaging portion 40 of the second rod portion 14 further includes a convexly-curved outer surface that corresponds to a concavely-curved inner surface of the retainer 17. In one variation, the outer surface of the engaging portion and the inner surface of the retainer 17 are spherical. In another variation, the outer edges of the engaging portion 40 are angled or sloped with respect to the longitudinal axis of the second rod portion 14. The slope is selected for customizing the angulation of the second rod portion 14 relative to the first rod portion 12 when connected therewith. In one variation, the second rod portion 14 further includes a beveled first end 36 having a radius of curvature. The bevel also plays a role in permitting the second rod portion 14 to angulate when disposed inside the retainer 17.

The second end 38 of the second rod portion 14 includes an anchor connecting portion 44 configured to be connected to an anchor. The anchor connecting portion 44 is sized and configured to be seated in a channel of a seat of a bone screw anchor for example. Any configuration for the second end 38 that is suitable for connection to an anchor or being received within the seat of an anchor is within the scope of the present invention and, for example, may include a pin-and-slot, snap-fit, compression-fit or other configuration including the type shown in FIG. 2a-2b for the anchor connecting portion 22 of the first rod portion 12.

The first rod portion 12, the second rod portion 14 or both may be curved to correspond to the lordotic curvature of a human spine. Preferably, the longer of the two rod portions is curved.

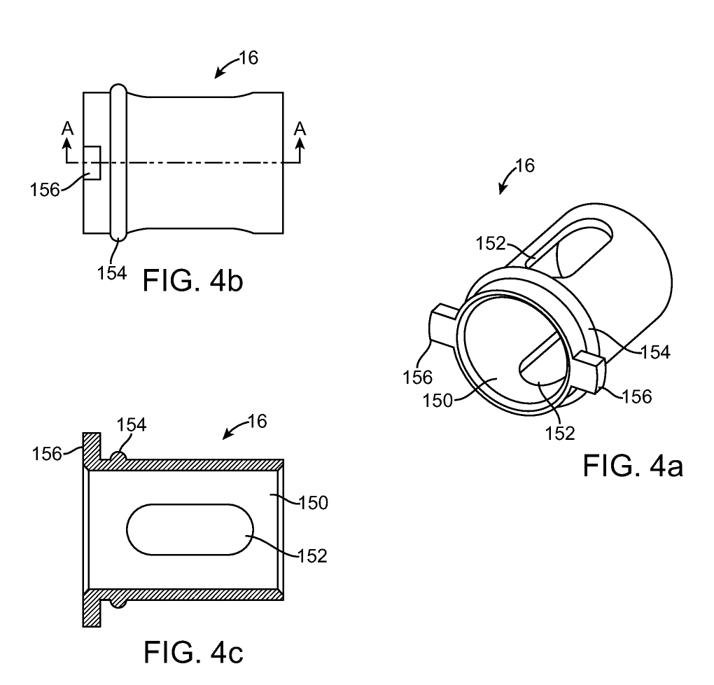

Referring now to FIG. 4a, there is shown a bias element 16 according to the present invention. In the variation shown, the bias element 16 is a polymeric bushing. The bias element 16 is made from any suitable material such as any polymer including but not limited to polyethylene, silicone or PEEK. The bias element 16 is sized to be receiving inside the retainer 17. The bias element 16 is cylindrical in shape with a central bore 150 opening at least at the first end of the bias element 16. The bias element 16 further includes a pair of oppositely-located and elongated pin apertures 152 configured for receiving the pin 31 of the first rod portion 12. The bias element 16 further includes a circumferential lip 154 and two projections 156 oppositely-located and extending from the outer surface of the bias element 16.

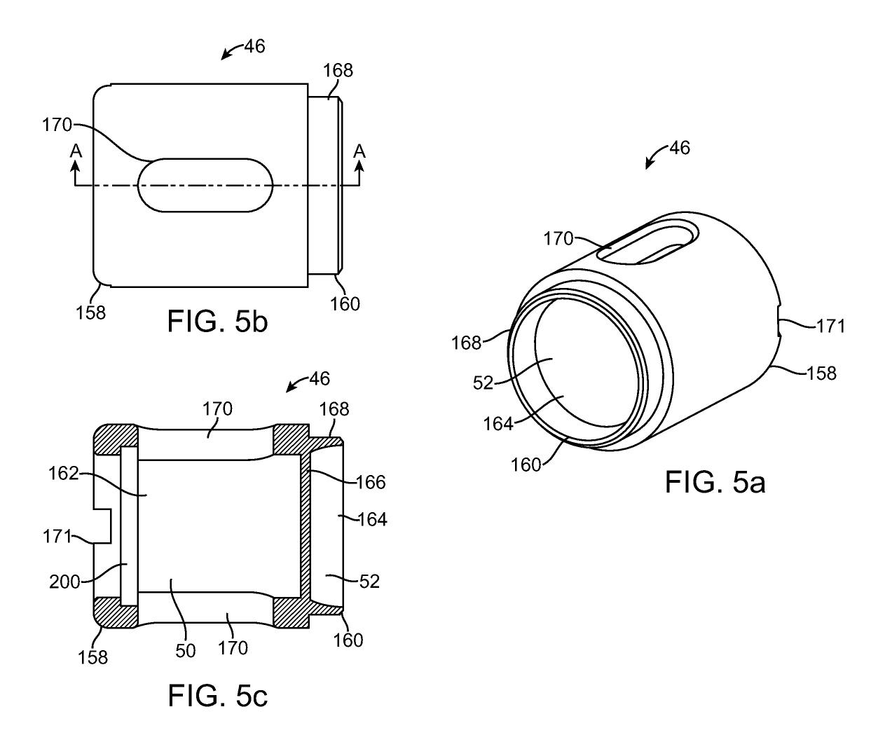

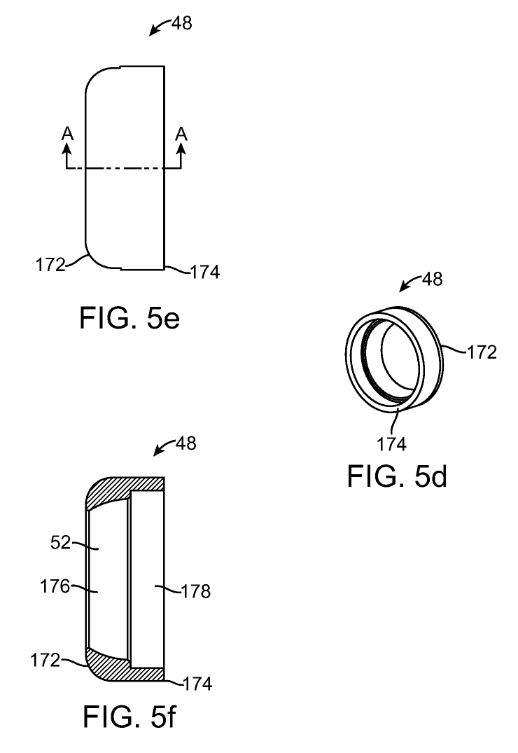

Turning now to FIGS. 5a-5f, there is shown the retainer 17 having a first piece 46 and a second piece 48 according to the present invention. The first piece 46 is depicted in FIGS. 5a-5c and the second piece 48 is depicted in FIGS. 5d-5f. Both the first piece 46 and the second piece 48 mate together to form the retainer 17. The retainer 17 is generally cylindrical and sleeve-like in shape. The retainer 17 is configured to encompass at least a portion of the first rod portion 12 and at least a portion of the second rod portion 14.

With particular reference now to FIGS. 5a-5c, the first piece 46 of the retainer 17 includes a first end 158 and a second end 160. The first piece 46 includes a first bore 162 opening at the first end 158 and extending inwardly of first piece 46. The first piece 46 includes a second bore 164 opening at the second end 160 and extending inwardly of the first piece 46. The first bore or first chamber 162 and the second bore 164 are separated by an inner wall 166. The inner surface of the first bore 162 includes a recess 200 configured to receive the lip 154 of the bias element 16. The second end 160 includes a collar portion 168 for connecting with the second piece 48 of the retainer 17. The first piece 46 also includes a pair of oppositely-located pin slots 170 configured to receive the pin 31 of the first rod portion 12 and configured to permit travel of the pin 31 within the slots 170. The first piece 46 further includes a pair of oppositely-located notches 171 configured to receive the projections 156 of the bias element 16. Accordingly, the first piece 46 defines a first receiving portion 50 at the first end 158 configured to receive therein at least a portion of the first rod portion 12 and, in particular, configured to receive at least a portion of the engaging portion 24 of the first rod portion 12 as shown in FIG. 1c. The second bore or second chamber 164 defines a portion of the second receiving portion 52 at the second end 160 that is configured, together with the second piece 48, to receive therein at least a portion of the second rod portion 14 and, in particular, configured to receive at least a portion of the engaging portion 40 of the second rod portion 14.

With particular reference now to FIGS. 5d-5f, the second piece 48 of the retainer 17 includes a first end 172 and a second end 174. The second piece 48 includes a first bore 176 opening at the first end 172 and extending inwardly of first piece 48. The second piece 48 includes a second bore 178 opening at the second end 174 and extending inwardly of the first piece 48. The second bore 178 is configured to cap the collar 168 of the first piece 46 of the retainer 17 to connect thereto. The first bore 176 and the second bore 178 are interconnected. The first bore 176 defines a portion of the second receiving portion 52 that is configured, together with the second piece 48, to receive therein at least a portion of the second rod portion 14 and, in particular, configured to receive at least a portion of the engaging portion 40 of the second rod portion 14 capturing the engaging portion 40 between the first piece 46 and the second piece 48. The first end 172 of the second piece 48 forms a constriction such that the bore opening at the first end 172 is smaller relative to the size of the disk-shaped engaging portion 40 of the second rod portion 14. The interior of the first bore 176 includes a concave, angled or spherical surface that substantially corresponds to the geometry of the engaging portion being received within the retainer 17. The second piece 48 further includes a pair of oppositely-located notches 180 (shown in FIG. 1a) configured for connection with a rod insertion instrument.

Referring back to FIGS. 1a-1c, the assembly of the dynamic rod 10 will now be discussed. The bias element 16 is placed inside the first receiving portion 50 of the first piece 46 of the retainer 17. The projections 156 of the bias element 16 are aligned with the notches 171 of the first piece 46 of the retainer 17 and the lip 154 is snapped into the recess 200. The pin apertures 152 of the bias element 16 are aligned with the pin slots 170 of the first piece 46 of the retainer 17. The second end 20 of the first rod portion 12 is inserted into the central bore 150 of the bias element 16 such that bias element 16 encompasses at least a portion of the first rod portion 12. Pin 31 is passed through one of the pin slots 170 on the first piece 46 and through a corresponding pin aperture 152 on the bias element 16 and press fit into bore 30 of the first rod portion 12 substantially flush to the retainer 17 as shown in FIG. 1b. The pin 31 extends laterally to the rod 10. In one variation, the pin 31 and the pin receiving apertures may be oriented by 90 degrees from the position shown in FIG. 1c such that the pin 31 extends in an anterior-to-posterior direction. The first rod portion 12 is allowed to move with respect to the retainer 17, said movement being limited by the pin 31 moving within the slot and abutting against the ends of pin slots 170. The second piece 48 of the retainer 17 is passed over the second end 38 of the second rod portion 14 and the first end 36 of the second rod portion 14 is inserted into the second bore 164 that forms part of the second receiving portion 52 in the first piece 46 of the retainer 17. The collar portion 168 of the first piece 46 is inserted into the second bore 178 of the second piece 48 and press-fitted and laser welded together to capture the engaging portion 40 of the second rod portion 14 within the second receiving portion 52 such that the second rod portion 14 is permitted to angulate or move polyaxially with respect to the retainer 17. In particular, the second rod portion 14 is capable of displacement from the longitudinal axis and in one variation, additionally capable of movement along the longitudinal axis relative to the retainer 17. The second rod portion 14 also rotates about the longitudinal axis. The first rod portion 12 is capable of movement along the longitudinal axis relative to the retainer 17 and constrained by the travel of the pin 31 within pin slots 170 and in one variation, additionally capable of slight displacement or angulation from the longitudinal axis. As shown in FIG. 1a, the dynamic rod 10 is assembled such that the longitudinal axis of pin 31 is substantially parallel to the longitudinal axis of the anchor connecting pins 26 and lie in the same plane such that the pin 31 advantageously does not interfere with insertion of the rod 10.

After the dynamic rod 10 is assembled, it is ready to be implanted within a patient and be connected to anchors planted in pedicles of adjacent vertebral bodies preferably in a manner such that the first rod portion 12 of the dynamic rod 10 illustrated in FIGS. 2a-2c is oriented cephalad and connected to the upper anchor and the second rod portion 14 is placed caudad and connected to the lower anchor. Because the first rod portion 12 includes an anchor connecting portion 22 configured such that connection with the anchor does not result in the rod extending cephalad beyond the anchor, this orientation and configuration of the dynamic rod is advantageous particularly because it avoids impingement of adjacent anatomy in flexion or in extension of the patient. Of course, in an alternative variation, the dynamic rod 10 is implanted into the patient such that the first rod portion 12 is oriented caudad and the second rod portion 14 is oriented cephalad.

Therefore, it is noted that the preferred implantation method and preferred orientation of the dynamic rod 10 is such that there is minimal or substantially no “overhanging” rod that extends cephalad beyond the upper anchor. Such orientation is achieved by the orientation of the rod during implantation as well as by the configuration of the anchor connecting portion 22, 44 of either one or both of the first rod portion 12 and second rod portion 14 such that the anchor connecting portion 22, 44 is configured such that there is substantially no overhang beyond the anchor when seated in the anchor.

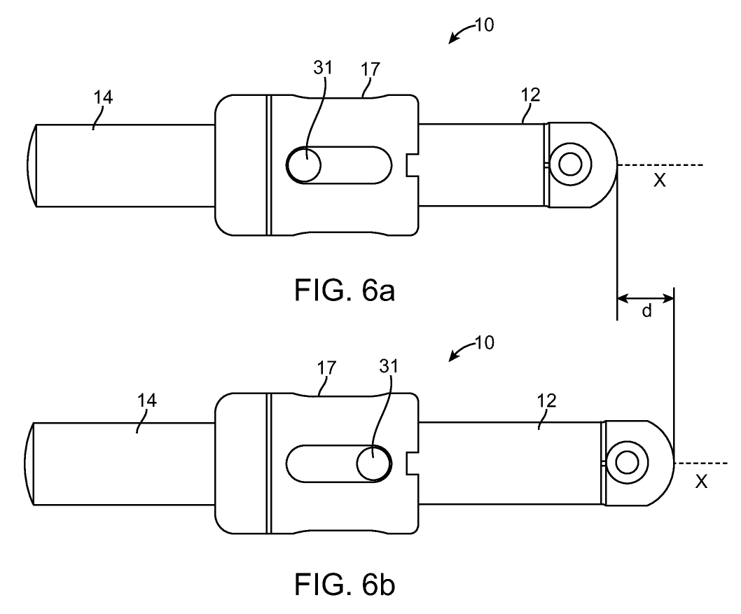

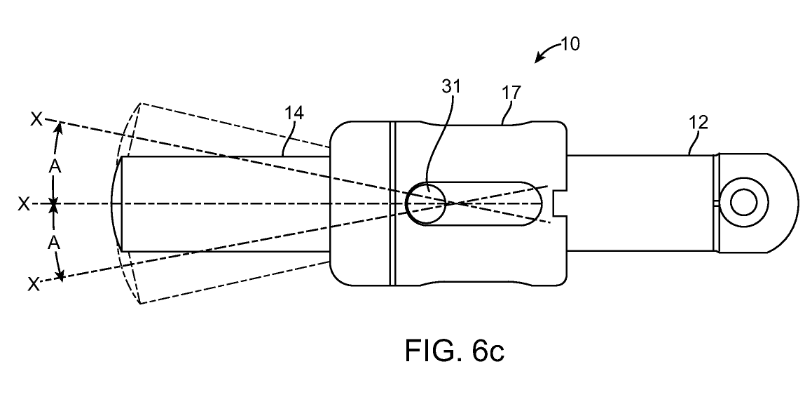

The implanted dynamic rod and anchor system fixes the adjacent vertebral bodies together in a dynamic fashion providing immediate postoperative stability and support of the spine. Referring now to FIGS. 6a-6c, the dynamic features of the dynamic rod 10 according to the present invention will now be discussed. In FIG. 6a, there is shown a dynamic rod 10 according to the present invention with the first rod portion 12 completely pushed into the retainer 17. FIG. 6b shows the first rod portion 12 extended outwardly along the longitudinal axis “x” relative to the retainer 17. As described above, the degree of longitudinal extension shown by “d” is determined by the travel of the pin 31 inside the pin slots 170 and is approximately between one and ten millimeters, and preferably approximately between two and five millimeters; however, the invention is not so limited and any suitable longitudinal extension is within the scope of the present invention. FIG. 6c illustrates displacement from the longitudinal axis or polyaxial angulation of the second rod portion 14 relative to the retainer by an angle “A”. Angle “A” is approximately between zero and twenty-five degrees, preferably between approximately eight and fifteen degrees with respect to the longitudinal axis “x”.

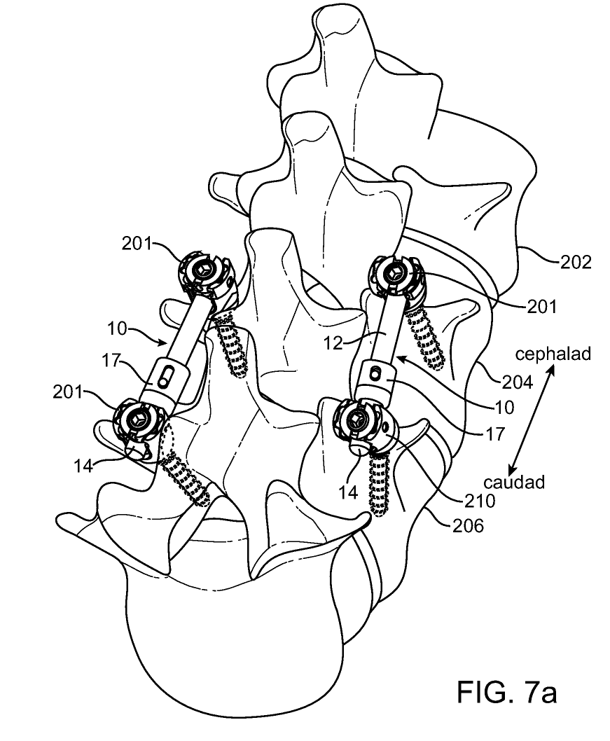

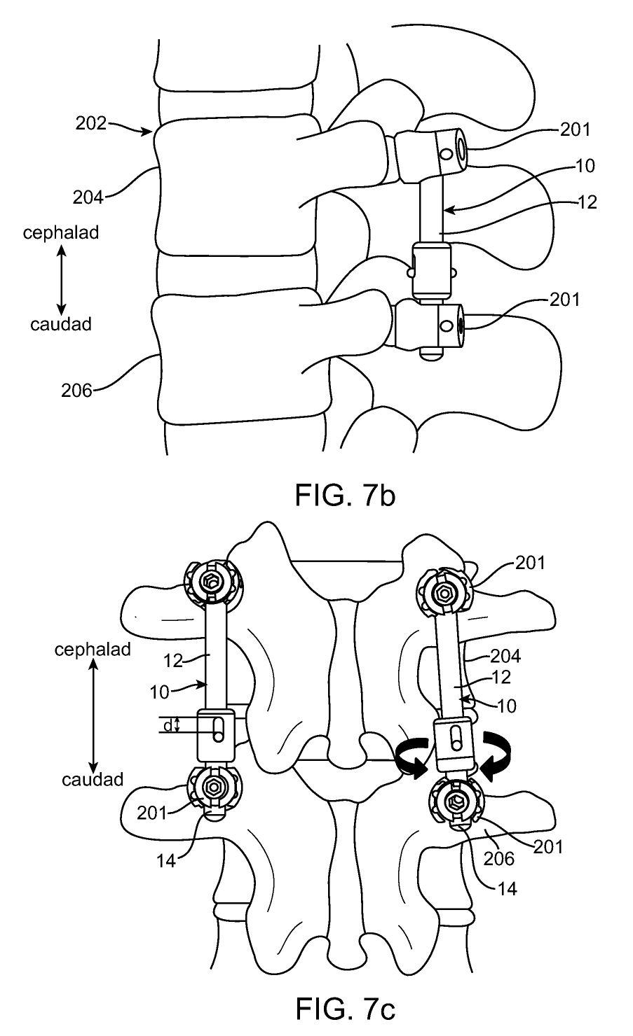

Hence, FIGS. 6a-6c illustrate that the dynamic rod 10 allows for movement described by a displacement from the longitudinal axis at one end of the rod 10 together with movement along the longitudinal axis at the other end of the rod. Referring now to FIGS. 7a-7c, there are shown different views of two dynamic rods 10 according to the present invention connected to anchor systems 201 implanted into a patient's spine 202 with a pair of anchor systems 201 in an upper vertebra 204 and a pair of anchor systems 201 in an adjacent lower vertebra 206 interconnected by two substantially parallel dynamic rods 10 according to the present invention. As seen in FIGS. 7a-7c, the first rod portion 12 is substantially longer than the second rod portion 14 and the rod 10 is oriented and implanted such that the retainer portion 17 is located closer to the lower vertebra 206 or lower anchor system 201. Such orientation advantageously places the angulating second rod portion 14 or fulcrum (with respect to the retainer 17) of the second rod portion 14 and the longitudinally moving first rod portion 12 closer to and more aligned with or adjacent to the lower facet joint, that is the facet joint formed by the inferior facet of the upper vertebra 204 and the superior facet of the lower vertebra 206, or pedicle, thereby advantageously off-loading the facet joint and placing the nexus of motion of the retainer as close as possible to the facet joint to more accurately mimicking the natural movement of the spine. Also, the second rod portion 14 rotates with respect to the retainer 17 through 360 degrees as shown in FIG. 7c. This rotation is also located at or close to the same facet joint, that is the facet joint formed by the inferior facet of the upper vertebra 204 and the superior facet of the lower vertebra 206, which thereby advantageously mimics the natural motion of the spine enabling the rod to carry some of the natural flexion and extension moments as well as rotation that the spine is subjected to. In cases where the dynamic rod 10 is subjected to a force perpendicular to the longitudinal axis of the rod 10 displacing one of the rod portions relative to the other rod portion away from the longitudinal axis, at least a portion of the bias element 16 may also be displaced from the longitudinal axis. The resulting displacement of the bias element 16 from the longitudinal axis establishes a biasing force exerted by the bias element in a direction opposite to its displacement to force the displaced rod portion toward a normal “relaxed” position substantially aligned with the longitudinal axis. The bias element 16 further cushions the shocks that the spine is subject to and prevents metal-on-metal contact of the first rod portion 12 and the retainer 17.

In another variation, and still referencing FIGS. 7a-7c, the dynamic rod 10 is configured such that the first rod portion 12 extending cephalad is further permitted to angulate relative to the retainer 17 in addition to moving longitudinally, extending in and out of the retainer 17, while the second rod portion 14 is permitted to angulate relative to the retainer 17 and in another variation in which the second rod portion 14 is locked in position.

In another variation, and still referencing FIGS. 7a-7c, the dynamic rod 10 is configured such that the second rod portion 14 extending caudad is fixed or lockable in position and does not angulate relative to the retainer while the first rod portion 12 is permitted to move longitudinally, extending in and out of the retainer 17. In another variation, the dynamic rod 10 is configured such that the first rod portion 12 is fixed or lockable in position and does not move longitudinally nor angulate while the second rod portion 14 is permitted to angulate relative to the retainer 17.

In another variation, the dynamic rod 10 is configured to be connected to the anchor systems 201 in reverse such that the second rod portion 14 is oriented cephalad and connected to the upper anchor system 201 of the upper vertebra 204 and the first rod portion 12 is placed caudad and connected to the lower anchor system 201 of the lower vertebra 206. The rod end connections are configured preferably such that there is no rod overhang beyond the upper anchor system 201 and that the joint of the first and second rod portions 12, 14 at the retainer 17 is located closer to the lower vertebra 206 or lower anchor system 201 relative to the upper anchor system 201. In such a variation, the second rod portion 14 is longer than the first rod portion 12 and the retainer 17 is located closer to and more aligned with or adjacent to the lower facet joint. In such a variation, the first rod portion 12 is movable along the longitudinal axis of the rod 10 within the constraints of the slots 170 and the second rod portion 14, which extends cephalad from the retainer 17, is permitted to angulate relative to the retainer 17. In one variation, the second rod portion 14 is fixed or lockable in position such that it cannot angulate relative to the retainer 17 while the first rod portion 12 extending caudad is permitted to move longitudinally, extending in and out of the retainer 17, and in another variation, additionally configured to angulate relative to the retainer 17 as well. In yet another variation, the dynamic rod 10 is configured such that first rod portion 12 extending caudad is permitted to angulate relative to the retainer 17 in addition to moving longitudinally and the second rod portion 14 extending cephalad is permitted to angulate relative to the retainer 17. In another variation, the dynamic rod 10 is configured such that the first rod portion 12 extending caudad is fixed or lockable and the second rod portion 14 extending cephalad remains permitted to angulate relative to the retainer 17. In another variation, both the first and second rod portions 12, 14 are configured to extend and angulate relative to the retainer 17 and in another variation both are configured to angulate relative to the retainer 17 only. In yet another variation, both the first and second rod portions 12, 14 are configured to move longitudinally relative to the retainer 17, extending in and out of the retainer 17. In such a variation, either the first or second or both rod portions 12, 14 may be configured to additionally angulate relative to the retainer 17.

In another variation that is applicable to all of the above iterations, the joint of the first and second rod portions 12, 14, or retainer 17 is located closer to the upper anchor system 201 implanted in the upper vertebra 204 relative to the lower anchor system 201 implanted in the lower vertebra 206. With the retainer 17 located closer to the superior facet joint of the motion segment, the dynamic rod 10 advantageously off-loads the facet joint and places the nexus of motion of the retainer as close as possible to the facet joint to more accurately mimic the natural movement of the spine. Also, for all of the iterations described above, angulation with respect to the retainer 17 is polyaxial angulation or otherwise limited and rotation relative to the retainer 17 is of either the first or second or both or none of the rod portions 12, 14 for any of the above variations. Furthermore, in another variation and for any of the above variations and iterations, the dynamic rod 10 is further configured to bias any angulation relative to the retainer 17. Such bias is provided by a spring (not shown) included inside the retainer 17 and configured such that when either the first or second or both rod portions is angulated or deflected away from the normal longitudinal position, the bias or spring provides a bias force to return the deflected rod back toward its normal position. Yet furthermore, in another variation and for any of the above variations and iterations any extension or longitudinal movement of a first or second or both rod portions 12, 14 relative to the retainer 17 may also be biased to return the movement back to its normal position. Such bias may also be provided by a spring located inside the retainer 17 and configured to provide an inward bias force against a rod portion that is extended outwardly relative to the retainer 17. Alternatively, such bias may also be provided by a spring located inside the retainer 17 and configured to provide an outward bias force against a rod portion that is extended inwardly relative to the retainer 17. Any combination of bias in a single dynamic rod 10 is within the scope of the present invention.

With two rods implanted in a patient's spine to stabilize two adjacent vertebral bodies with one rod on each side of the spinous process as shown in FIGS. 7a and 7c, the motion of the rod(s) relative to the anatomy will now be discussed. When the patient bends forward in flexion, the rod 10 angulates and extends longitudinally. In particular, when the patient bends forward in flexion, the second rod portion 14 angulates, pivots or otherwise rotates relative to the first rod portion 12 to form an acute angle therebetween with the rod 10 angled forward. Also in forward flexion, the first rod portion 12 extends longitudinally outwardly relative to the retainer 17 increasing the overall length of the rod 10. Hence, the rod matches the anatomical motion of the spine, providing extension and angulation of the rod when the patient bends forward. The rod of the present invention also advantageously provides dynamic stabilization when the patient bends backwards in extension. In extension, the rod angulates backwards. In particular, the second rod portion 14 angulates, pivots or otherwise rotates relative to the first rod portion 12 to form an acute angle therebetween in the opposite direction than in forward flexion such that the rod 10 is angled backwards. In extension, the first rod portion 12 typically does not extend longitudinally outwardly relative to the retainer but may extend inwardly into the retainer to reflect the decrease in length of the entire rod in extension relative to longer length of the entire rod in flexion. Of course, the longitudinal travel of the first rod portion 12 is limited or controlled by the slot with the pin 31 traveling therein providing dynamic limits. Furthermore, the angulation of the rod is constrained by the geometric construct of the retainer, thereby, providing controlled dynamic stabilization. The rod 10 of the present invention also advantageously provides dynamic stabilization when the patient bends side-to-side in lateral bending. In the system of two rods implanted on either side of the spinous processes, the rod 10 positioned closer to or near the direction of the bending or inside the curvature of the bend will angulate in the direction of the bend whereas the rod 10 positioned on the far side or on the outside of the curvature of the bend will angulate in the direction of the bend and additionally extend longitudinally increasing the overall length of the rod 10. In particular, when the patient bends to one side, the second rod portion 14 of the rod 10 on the side of the bend angulates, pivots, or rotates toward the direction of the bend and the second rod portion 14 of the rod 10 on the far side of the bend also angulates, pivots, or rotates in the direction of the bend and the first rod portion 12 of the rod 10 on the far side of the bend further extends longitudinally increasing the overall length of the rod 10 to accommodate the natural extension of the spine on the far side of the bend relative to the near side of the bend which experiences a contraction which may cause the first rod portion 14 of the rod 10 on the near side of the bend to move inwardly into the retainer, decrease in length to accommodate the natural contraction of the spine on the near side of the bend.

The dynamic rod 10 of the present invention is suitable for treating indications including but not limited to facet degeneration, nerve root impingement, morbid obesity, previous abdominal surgery, spondylolisthesis, spinal stenosis, scoliosis, osteoporosis, and deficiency of posterior elements. From the above, it is evident that the present invention can be used to relieve pain caused by spinal stenosis in the form of, by way of example only, central canal stenosis or foraminal stenosis, degenerative disc disease, spondylolisthesis, spinal deformities, fracture, pseudarthrosis and tumors.

All publications mentioned herein are incorporated herein by reference to disclose and describe the methods and/or materials in connection with which the publications are cited. The preceding illustrates the principles of the invention. It will be appreciated that those skilled in the art will be able to devise various arrangements which, although not explicitly described or shown herein, embody the principles of the invention and are included within its spirit and scope.

We claim:

1. A dynamic rod comprising:
a first rod portion;
a second rod portion dynamically connected to the first rod portion; and
a retainer having a first chamber configured to retain an end of the first rod portion and a second chamber configured to retain an end of the second rod portion;
wherein the first and second chambers are separated by a wall formed in the retainer.

2. The dynamic rod of claim 1 wherein the first rod portion is configured to move relative to the retainer along the longitudinal axis of the dynamic rod.

3. The dynamic rod of claim 1 wherein the second rod portion is configured to angulate polyaxially relative to the retainer.

4. The dynamic rod of claim 1 wherein the first rod portion is configured to move relative to the retainer along the longitudinal axis of the dynamic rod and the second rod portion is configured to angulate polyaxially relative to the retainer.

5. The dynamic rod of claim 1 further including a pin passed through at least one slot formed in the retainer and connected to the first rod portion such that movement of the first rod portion relative to the retainer is limited by the pin moving within the at least one slot.

6. The dynamic rod of claim 1 further including a bias element located in the retainer between the first rod portion and the retainer and configured to bias movement of the first rod portion relative to the retainer.

7. The dynamic rod of claim 1 further including a bias element located in the retainer between the second rod portion and the retainer and configured to bias movement of the second rod portion relative to the retainer.

8. The dynamic rod of claim 1 wherein the first rod portion is longer than the second rod portion.

9. A dynamic rod comprising:
a first rod portion;
a second rod portion dynamically connected to the first rod portion;
a retainer configured to retain an end of the first rod portion and an end of the second rod portion; and
a pin passed through at least one slot formed in the retainer and connected to the first rod portion such that longitudinal movement of the first rod portion relative to the retainer is limited to within the at least one slot.

10. The dynamic rod of claim 9 wherein the second rod portion is configured to angulate relative to the retainer.

11. The dynamic rod of claim 9 wherein the first rod portion is configured to angulate relative to the retainer.

12. A dynamic rod comprising:
a first rod portion;
a second rod portion dynamically connected to the first rod portion;
a retainer configured to retain an end of the first rod portion and an end of the second rod portion; and
wherein the first rod portion is configured for longitudinal movement relative to the retainer and the second rod portion is configured to angulate polyaxially relative to the retainer.

13. The dynamic rod of claim 12 wherein the first rod portion is oriented cephalad and the second rod portion is oriented caudad when implanted in a patient.

14. The dynamic rod of claim 12 wherein the first rod portion is longer than the second rod portion.

15. The dynamic rod of claim 12 further configured such that the retainer is proximate to the lower facet joint when implanted into a patient.

16. The dynamic rod of claim 12 wherein the first rod portion is further configured to angulate polyaxially.

17. The dynamic rod of claim 12 wherein the second rod portion is longer than the first rod portion.

18. The dynamic rod of claim 12 further configured such that the retainer is proximate to the upper facet joint when implanted into a patient.

19. A dynamic rod comprising:
a first rod portion;
a second rod portion dynamically connected to the first rod portion at a joint; and
wherein the first rod portion is longer than the second rod portion.

20. The dynamic rod of claim 19 wherein the first rod portion is configured such that the joint is proximate to a facet joint of the spine when implanted into a patient.
